(19) United States
(12) Patent Application Publication
(10) Pub. No.: US 2004/0137035 A1
(43) Pub. Date: Jul. 15, 2004

(54) Granulated crystal compilation composed of pure organic minerals used in all food applications resulting in energy supplementation, diet and weight regulation, and medicinal effects

(76) Inventor: Daniel W. Hassani, Lake Elsinore, CA (US)

Correspondence Address: DANIEL W. HASSANI, 18495 CRESTLINE DR., LAKE ELSINORE, CA, 92532, US

(21) Appl. No.: 10/252,928

(22) Filed: Jan. 9, 2003

(51) Int. Cl. A61K 35/78; A61K 47/00

(52) U.S. Cl. 424/439; 424/725

(57) ABSTRACT

A granulated crystal compilation composed of pure organic minerals used in all food applications resulting in energy supplementation, diet and weight regulation, and medicinal effects.

### BACKGROUND OF THE INVENTION

This invention relates to compositions of various minerals that produce advantageous beneficial effects in the forms of energy derivation, proper nutrition cognizance, and energy supplementation.

The human body has a natural process in which it creates, utilizes, recycles, and reintegrates energies in the forms of complex molecules (in this aspect we are looking at the specific dormant energy containment structures known as complex carbohydrates or complex sugars). As one consumes food the bodies digestive system breaks it down into complexes later used for energy.

When food is consumed the body will obtain a certain amount of energy from proper synthesis of it into usable elements. It is possible to consume food that takes more energy to synthesis than the total energy received from the process. Then depending on metabolism and lifestyle the energy obtained would either be stored in fatty forms or used for energy.

The citric acid cycle is a reoccurring cycle which takes complex molecules and manipulates them into usable energies and then recycles the leftover energy into usable energy again in return we have a constant supply of expendable energy, as long as there are enough of those specific complex molecules present to initiate the reaction cycle process.

Citric acid (anhydrous/hydrated), as well as all citrates contain the similar molecular composition and structure as the molecule citrate. Citrate is a specific initiating molecule present in the citric acid cycle. In fact citrate is the initiating molecule resulting in constant supply of energy as long as citrate is being introduced to the cycle.

Citric compound* contains the active ingredient/s (Components A) and when consumed and digested will release citric acid and citrate into the body for beneficial effects explained throughout this application.

### SUMMARY OF THE INVENTION

It is the objective of this product to provide a healthy all natural solution to modem day problems in the areas of energy, diet, and medicine. The precise incrementation of the selected minerals allows for a wide variety of benefits.

Blood and Circulatory Benefits Produced From Citric Compound*.

The consumption of the citric compound* results in the strengthening, filtering, and optimizing of the bodies circulatory system. The introduction of a citric compound* into blood by ingestion of “citric acid” has the positive effects of destroying the adipose tissue and cholesterol build-up in blood stream. Implementation of the citric compound* into the body will result in the cleansing of the complete circulatory system from impurities

known such as viral, bacterial, and Other contaminants***. Consuming the citric compound* will reduce the amount of cholesterol present in the body as well as purify the blood and optimize the entire circulatory system.

Diet Advantages and Weight Loss Effects From Citric Compound*.

While the circulation of the citric compound* in the body destroys cholesterol in the blood vessels it is also present throughout the entire bodies bloodstream. There it will eventually be distributed to areas of adipose concentration eliminating adipose tissue prior to solidification in concentrated areas. The resulting effect is the overall body fat percentage decreases. As long as the citric compound* is in circulation and in contact with adipose complexes it will eliminate the adipose complex until the concentration of the citric compound* decreases to the point where the neutralization reaction with the adipose tissue ends. Thus if the citric compound* is ingested with fats and/or fatty acids the resulting adipose tissue stored would in fact be lower than if not taken with the citric compound*.

Energy Supplementation From Citric Compound*.

Depending on the type food consumed the percentage of energy gained from consumption varies. The intake of the citric compound* will result in a high energy gain due to release of citrate. The citric compound* aids in the digestive process and allows for less energy used during digestion thus conserving energy. The digestion of the citric compound* will result in energy received by cellular synthesis (FIG. 8), resulting in an overall body loss of adipose tissue (fat ) decreasing the body fat percentage.

Citric compound*—pertain to any combination or variation of the chemical molecules specified in the First major Component “A”

Other contaminants***—pertain to contaminants in body which are rendered inactive due to ph neutralization when in contact with the citric compound*.

### DETAILED DESCRIPTION

The mineral composition consists of three major components in any combination/variation.

Component A:

The “Component A” category are chemicals that makeup any percentage of the final compound from 1%-99.9% and is composed of any of the following compounds in any combination, arrangement, and/or variation.

1. Citric Acid (Anhydrite)

2. Citric Acid (Hydrated)

3. Sodium Citrate

4. Monosodium Citrate

5. Trisodium Citrate

6. Potassium Citrate

7. MonoPotassium Citrate

8. TriPotassium Citrate

Component B:

The “Component B” category are chemicals that makeup any percentage of the final compound from 1%-80% and is composed of Potassium Chloride.

Component C:

The “Component C” category makeup any percentage of the final compound from 1%-50% and is composed of Sodium Chloride.

Optional Component D:

There can be minor components present in the total compound and still have the same effects from the First major Components as long as the percentage of the minor components does not exceed 10% in order to sustain productivity.

All compounds named above are of F.D.A. food grade. The preferred arrangement of compounds for food applications is 50%-99.9% Component A, 0%-75% Second Major Component, and 0%-25% Third Major Component.

Detailed descriptions of the tables contained in this application.

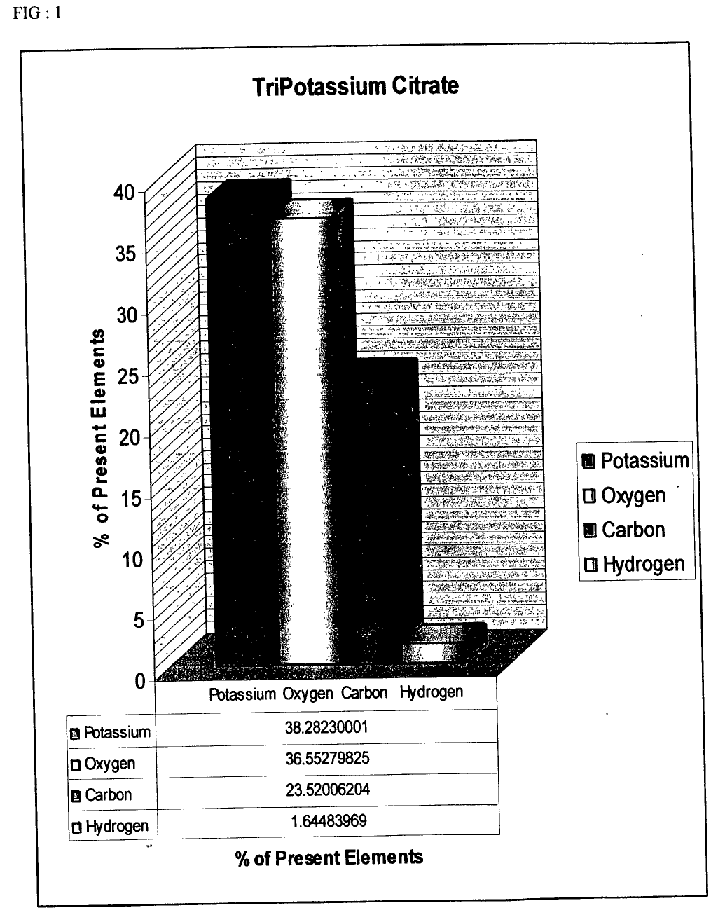

FIG. 1: Contains a detailed explanation of the percentages of specific elements in Tripotassium Citrate.

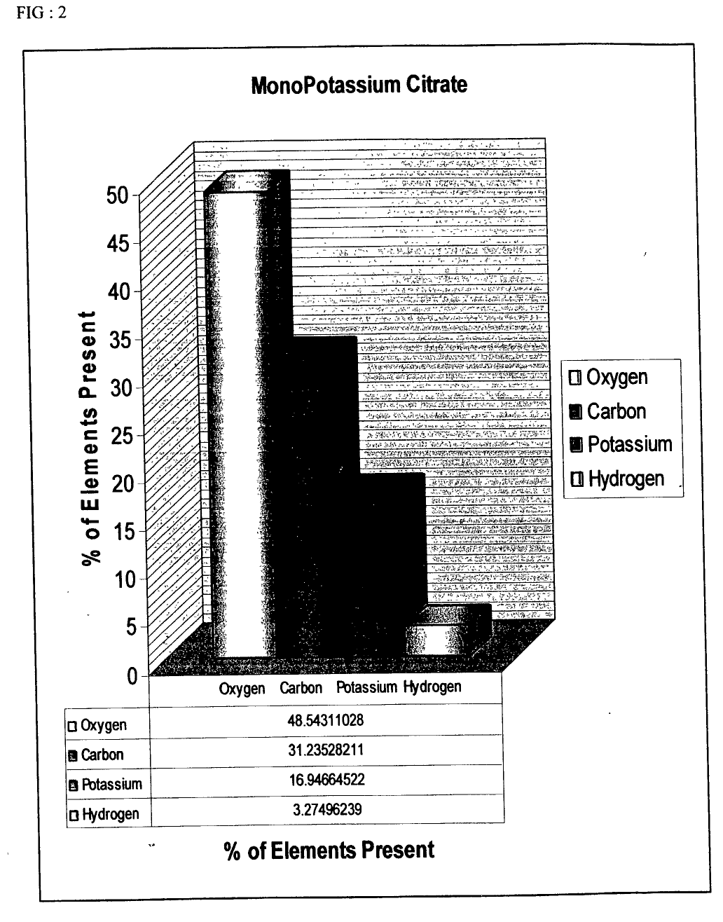

FIG. 2: Contains a detailed explanation of the percentages of specific elements in Monopotassium Citrate.

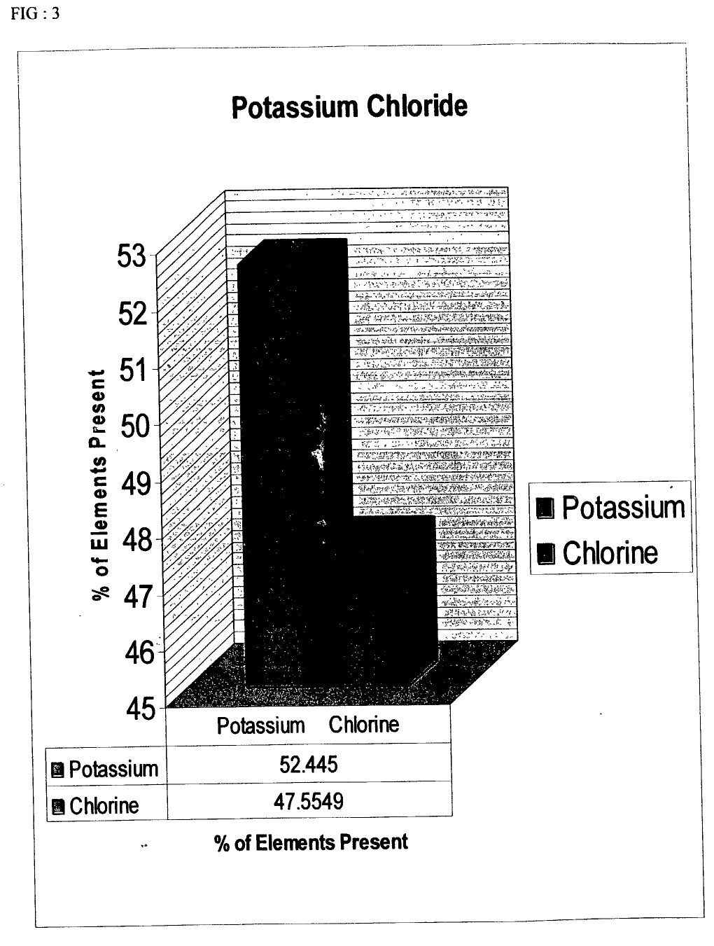

FIG. 3: Contains a detailed explanation of the percentages of specific elements in Potassium Chloride.

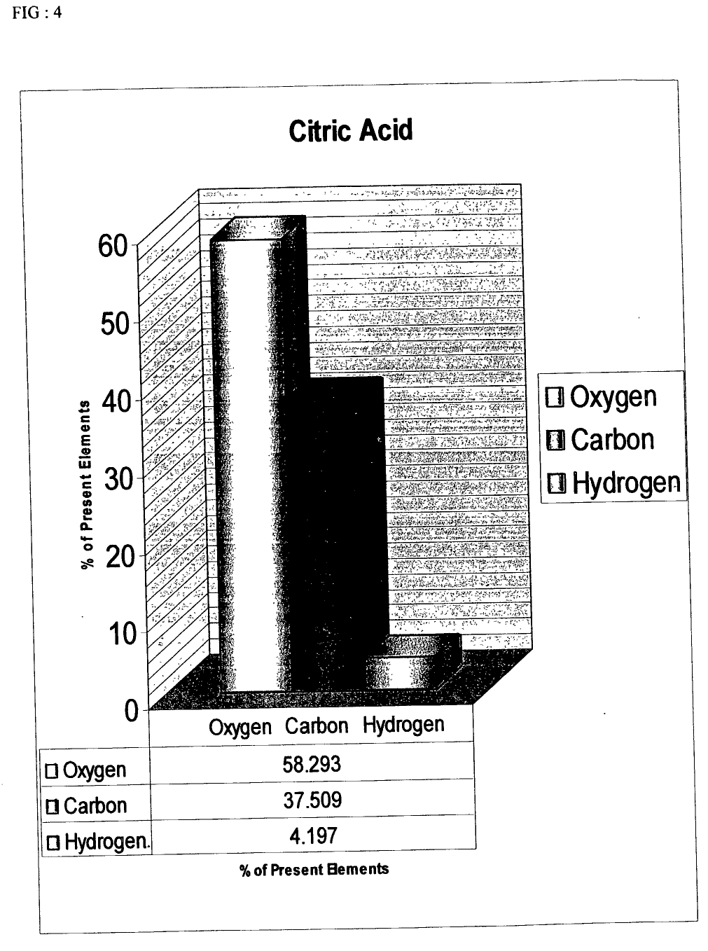

FIG. 4: Contains a detailed explanation of the percentages of specific elements in Citric Acid.

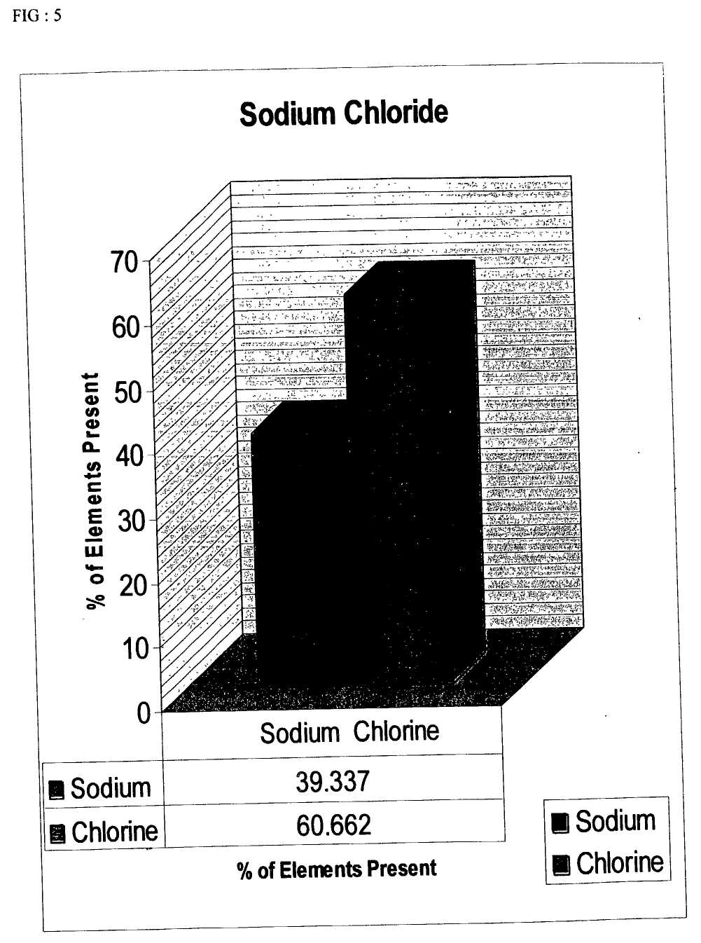

FIG. 5: Contains a detailed explanation of the percentages of specific elements in Sodium Chloride.

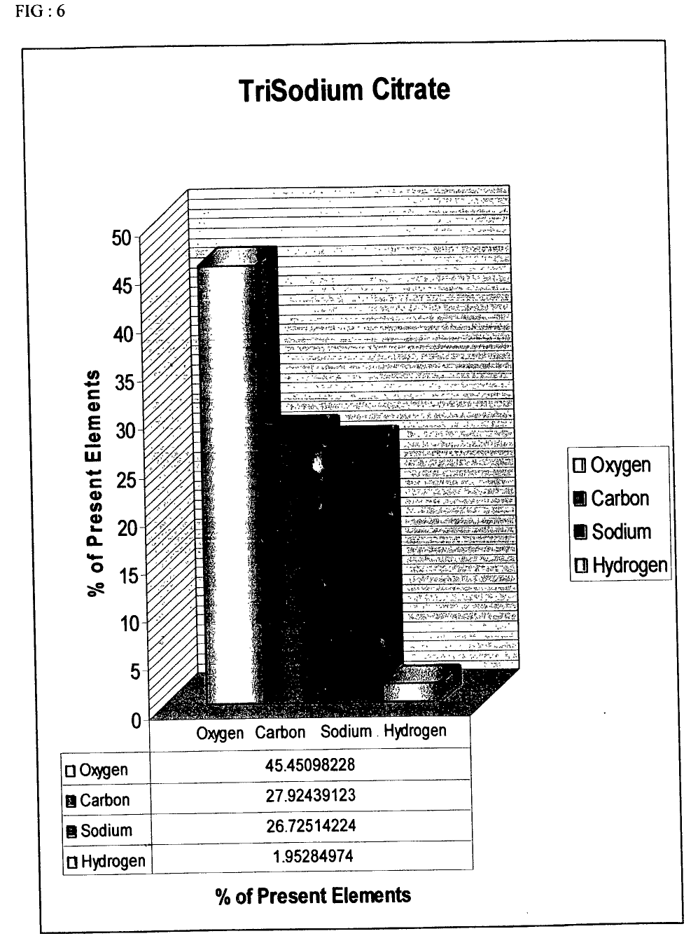

FIG. 6: Contains a detailed explanation of the percentages of specific elements in TriSodium Citrate.

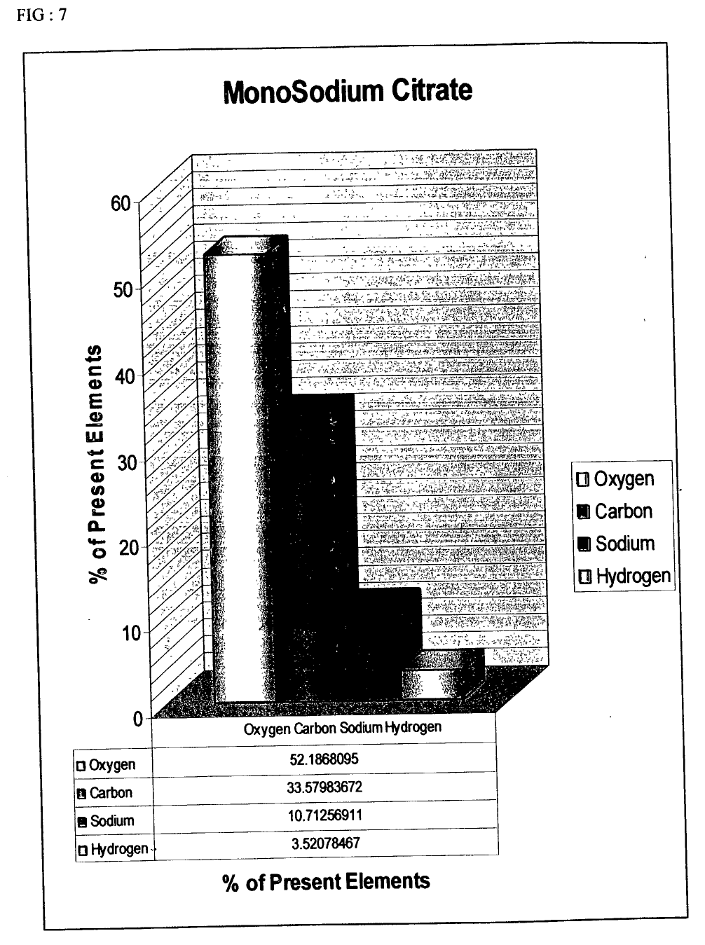

FIG. 7: Contains a detailed explanation of the percentages of specific elements in MonoSodium Citrate.

FIG. 8: Describes an example of energy synthesis through breaking down the molecule of Citric Acid.

We claim all the following objectives of this invention as direct results brought forth by the oral consumption of the citric compound* introduced and explained in the “detailed description” and throughout this application:

1. It is the objective of this invention to strengthen/optimize the condition and functionality of the blood cells throughout the body by filtering out impurities and/or reconditioning the blood cells and plasma.

2. It is the objective of this invention to dissolve complex fatty acids, cholesterol, and all forms of fats and oils present in all blood vessels.

3. It is the objective of this invention to begin destroying fats upon contact with them.

4. It is the objective of this invention to reduce the amount of fat present in food substance during and prior to digestion and/or ingestion.

5. It is the objective of this invention to reduce the amount of fats and oils present in the entire digestive tract.

6. It is the objective of this invention to reduce the overall body fat percentage.

7. It is the objective of this invention to be a source of energy.

8. It is the objective of this invention to reduce the amount of food consumed by suppling energy of its own to compensate the loss due to the decreased food intake.

9. It is the objective of this invention to conserve existing energy by breaking up consumed food allowing for less energy expended on digestion.

10. It is the objective of this invention to aid in the digestion process allowing for increased energy obtained by natural ingestion of consumed foods.
